(19) United States
(12) Patent Application Publication
(10) Pub. No.: US 2024/0050114 A1
(43) Pub. Date: Feb. 15, 2024

(54) SURGICAL INSTRUMENTS HAVING REPLACEABLE SEAL PLATES

(71) Applicant: Covidien LP, Mansfield, MA (US)

(72) Inventors: Rajanikanth Mandula, Hyderabad (IN); Ravi Sekhar Gutti, Hyderabad (IN)

(21) Appl. No.: 17/884,158

(22) Filed: Aug. 9, 2022

(51) Int. Cl. A61B 17/28; A61B 18/14

(52) U.S. Cl. CPC A61B 17/282; A61B 18/1445; A61B 2018/0063

(57) ABSTRACT

A surgical instrument includes a housing, a shaft, an end effector, a lead wire, and a first replaceable seal plate. The shaft extends distally from the housing. The end effector is supported adjacent a distal end of the shaft and includes a first jaw member and a second jaw member. One or both of the first jaw member or the second jaw member is movable relative to the other between an open position and an approximated position. The lead wire extends at least partially through the shaft and is adapted to connect to a source of electrosurgical energy. The first replaceable seal plate is configured for selective engagement with the first jaw member and configured for selective engagement with the lead wire. The first replaceable seal plate includes an electrically conductive portion and is configured for contacting tissue when engaged with the first jaw member.

### BACKGROUND

### Technical Field

The present disclosure relates to surgical instruments and, more particularly, to surgical instruments having replaceable seal plates.

### BACKGROUND OF RELATED ART

Open, elongated or laparoscopic surgical instruments having jaw members are often used during a variety of surgical to grasp, clamp, seal, join and/or divide tissue. During certain electrosurgical procedures, dead tissue known as eschar may build up on the seal plates of the jaw members of the surgical instruments. The eschar build-up on the seal plates may not be desirable for a variety of reasons, and may lead the surgeon or nurse to clean the seal plates during the surgical procedure. The cleaning of the seal plates can be time consuming and may not be as efficient in removing eschar as desired.

Additionally, seal plates may become damaged during shipping or use, for instance, thereby possibly reducing their effectiveness.

### SUMMARY

As used herein, the term “distal” refers to the portion that is being described which is further from an operator, e.g., a surgeon or a surgical robot, while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a surgical instrument including a housing, a shaft, an end effector, a lead wire, and a first replaceable seal plate. The shaft extends distally from the housing. The end effector is supported adjacent a distal end of the shaft and includes a first jaw member and a second jaw member. One or both of the jaw members is movable relative to the other between an open position and an approximated position. The lead wire extends at least partially through the shaft and is adapted to connect to a source of electrosurgical energy. The first replaceable seal plate is configured for selective engagement with the first jaw member and configured for selective engagement with the lead wire. The first replaceable seal plate includes an electrically conductive portion and is configured for contacting tissue when engaged with the first jaw member.

In aspects of the disclosure, a first distal end of the lead wire includes a first crimp contact. In aspects, a finger extends proximally from a proximal portion of the first replaceable seal plate and is configured for pivotal engagement with the first crimp contact.

In another aspect of the disclosure, the first jaw member includes a shell defining a cavity, and the first replaceable seal plate is selectively positionable at least partially within the cavity. In aspects, the shell of the first jaw member includes a plurality of lips configured to engage the first replaceable seal plate. In aspects, the first replaceable seal plate includes a plurality of grooves. Each lip of the plurality of lips of the shell of the first jaw member is configured to engage one groove of the plurality of grooves of the first replaceable seal plate. In aspects, one or more grooves of the plurality of grooves of the first replaceable seal plate is defined within a first lateral side of the first replaceable seal plate, and one or more grooves of the plurality of grooves of the first replaceable seal plate is defined within a second lateral side of the first replaceable seal plate. In aspects, a distal end of the first replaceable seal plate defines a detent configured to facilitate disengaging the first replaceable seal plate from the first jaw member.

In still other aspects, the surgical instrument includes a second replaceable seal plate configured for selective engagement with the second jaw member and configured for selective engagement with the lead wire. In aspects, a second distal end of the lead wire includes a second crimp contact. In aspects, a proximal portion of the second replaceable seal plate is configured for pivotal engagement with the second crimp contact.

In yet other aspects, the first jaw member includes a shell that defines a slit adjacent the first crimp contact. A finger extending proximally from a proximal portion of the first replaceable seal plate is configured to selectively extend at least partially through the slit.

The present disclosure also relates to an end effector for use with a surgical instrument. The end effector includes a first jaw member, a second jaw member, a lead wire, a first replaceable seal plate, and a second replaceable seal plate. One or both of the first jaw member or the second jaw member is movable relative to the other between an open position and an approximated position. The lead wire is disposed in mechanical cooperation with the first jaw member and the second jaw member, and is adapted to connect to a source of electrosurgical energy. The first replaceable seal plate is configured for selective engagement with the first jaw member and is configured for selective engagement with the lead wire. The second replaceable seal plate is configured for selective engagement with the second jaw member and is configured for selective engagement with the lead wire.

In aspects of the disclosure, a first distal end of the lead wire includes a first crimp contact associated with the first jaw member, and a second distal end of the lead wire includes a second crimp contact associated with the second jaw member. In aspects, a finger extends proximally from a proximal portion of the first replaceable seal plate and is configured for pivotal engagement with the first crimp contact. In aspects, a finger extends proximally from a proximal portion of the second replaceable seal plate and is configured for pivotal engagement with the second crimp contact.

In further aspects of the disclosure, the first replaceable seal plate is configured to selectively engage the first jaw member with a snap-fit connection, and the second replaceable seal plate is configured to selectively engage the second jaw member with a snap-fit connection. In aspects, a first lateral wall of the first replaceable seal plate and a first lateral wall of the second replaceable seal plate each include a groove. The first jaw member and the second jaw member each include one or more lips. The lip of the first jaw member is configured to selectively engage the groove of the first replaceable seal plate, and the lip of the second jaw member is configured to selectively engage the groove of the second replaceable seal plate. In aspects, a distal end of the first replaceable seal plate defines a first detent configured to facilitate disengaging the first replaceable seal plate from the first jaw member, and a distal end of the second replaceable seal plate defines a second detent configured to facilitate disengaging the second replaceable seal plate from the second jaw member.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a surgical instrument provided in accordance with the present disclosure;

FIG. 2 is a side perspective view of a proximal portion of the surgical instrument of FIG. 1 with a portion of the housing removed to illustrate internal features therein;

FIG. 3 is an exploded, perspective view of the surgical instrument of FIG. 1;

FIG. 4 is a perspective view of an end effector of the surgical instrument of FIG. 1;

FIG. 5 is a perspective view of the end effector of FIG. 4 illustrating seal plates separated from jaw members of the end effector;

FIG. 6 is an enlarged view of the area of detail indicated in FIG. 4;

FIG. 7 is a perspective view of the end effector of FIG. 4 with the seal plates removed;

FIG. 8 is an enlarged view of the area of detail indicated in FIG. 7;

FIG. 9 is a perspective view of the end effector of FIG. 4 illustrating one seal plate being removing from its associated jaw member;

FIG. 10 is a perspective view of the end effector of FIG. 4 illustrating one seal plate disengaged from its associated jaw member;

FIG. 11 is a perspective view of the end effector of FIG. 4 illustrating one seal plate removed, and an overmold portion of one jaw member in phantom;

FIG. 12 is an enlarged view of the area of detail indicated in FIG. 11;

FIGS. 13 and 14 are perspective views of the end effector of FIG. 4 illustrating one seal plate being inserted into its associated jaw member;

FIG. 15 is an enlarged view of the area of detail indicated in FIG. 14;

FIG. 16 is a cross-sectional view of the end effector of FIG. 4 taken along line 16-16 in FIG. 14;

FIG. 17 is a perspective view of an end effector in accordance with a second embodiment of the present disclosure illustrating a replaceable seal plate separated from one of the jaw members;

FIG. 18 is an enlarged, perspective view of a distal end of a jaw member and a proximal end of the replaceable seal plate of FIG. 17;

FIG. 19 is a perspective view of a lower portion of the replaceable seal plate engaged with the jaw member of FIG. 17;

FIG. 20 is a cross-sectional view of the end effector and replaceable seal plate of FIG. 17 taken along line 20-20 in FIG. 19 illustrating a locking mechanism in a locked position; and

FIG. 21 is a cross-sectional view of the end effector and replaceable seal plate of FIG. 17 taken along line 20-20 in FIG. 19 illustrating a locking mechanism in an unlocked position.

### DETAILED DESCRIPTION

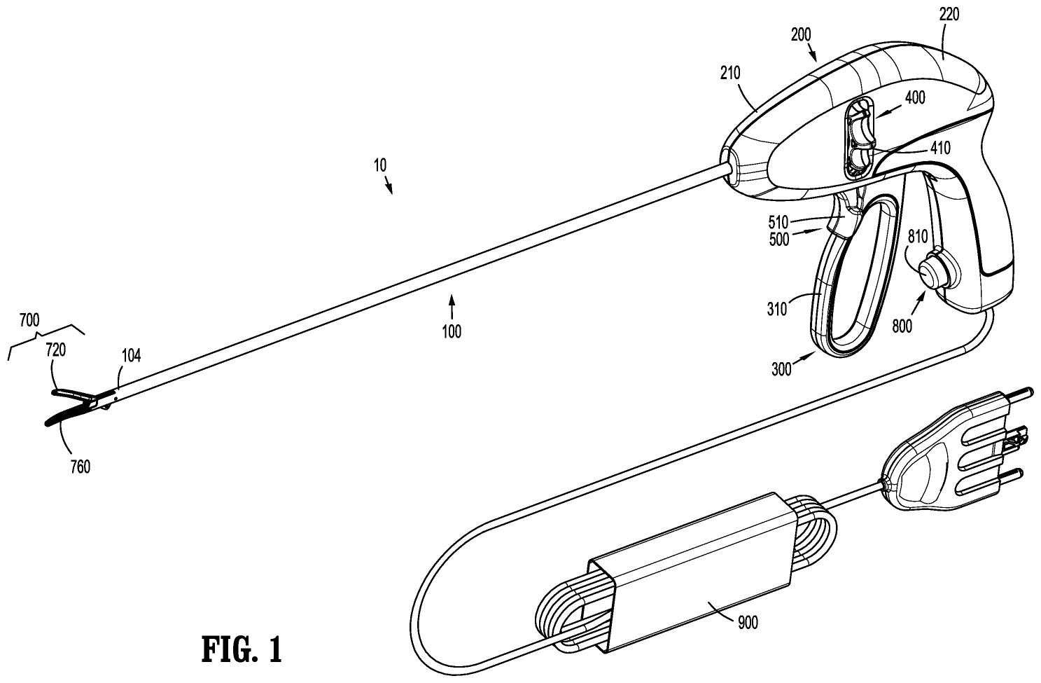

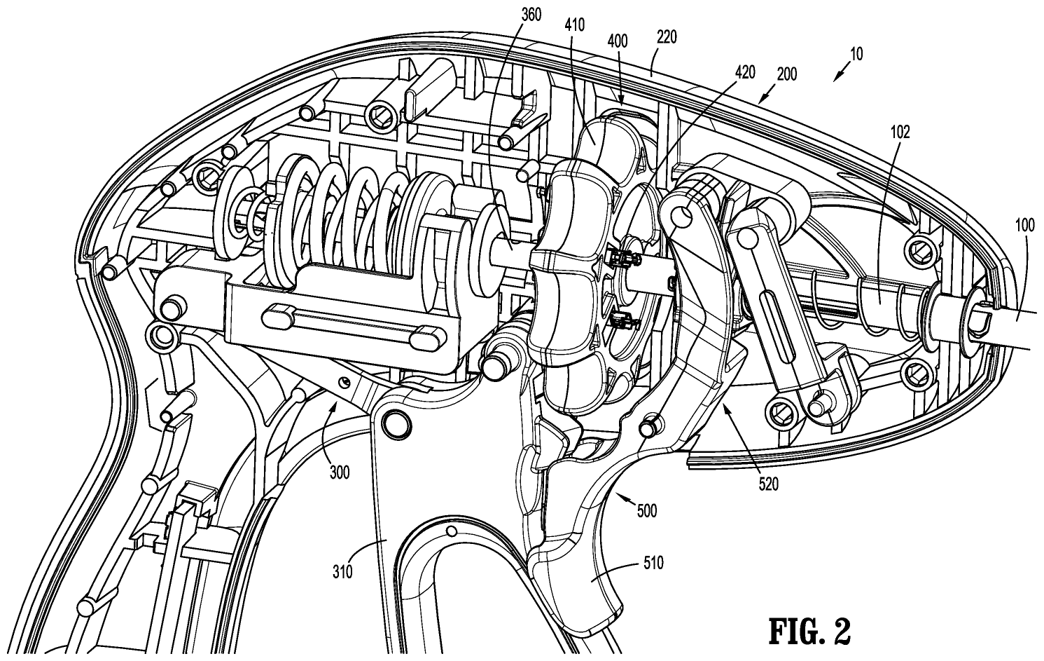

Referring generally to FIG. 1-3, a surgical instrument provided in accordance with the present disclosure and configured for grasping, treating, and/or dividing tissue is shown generally identified by reference numeral 10. Surgical instrument 10 includes a shaft 100, a housing 200, a drive assembly 300, a rotation assembly 400, a trigger assembly 500, a knife assembly 600, an end effector 700, an activation assembly 800, and a cable 900.

Shaft 100 extends distally from housing 200 and supports end effector 700 at a distal portion 104 thereof. Housing 200 of surgical instrument 10 includes first and second housing parts 210, 220 secured to one another, e.g., via ultrasonic welding, to operably support and/or enclose various components of surgical instrument 10 within housing 200.

Drive assembly 300 includes, among other components, a movable handle 310 that is operably coupled to a drive sleeve 360 within housing 200. Drive sleeve 360 extends from housing 200 through shaft 100 to operably couple with end effector 700 such that pivoting of movable handle 310 relative to housing 200 between an open or un-actuated position, wherein movable handle 310 is farther spaced-apart from the fixed handle portion of housing 200, and an actuated position, wherein movable handle 310 is more closely approximated relative to the fixed handle portion of housing 200, pivots jaw member 720 (or both jaw members 720, 760) of end effector 700 relative to the other jaw member 760 of end effector 700 from a spaced-apart position to an approximated position. Movable handle 310 is further movable towards the fixed handle portion of housing 200 to an activated position to activate activation button 810 of activation assembly 800, although a separate hand switch disposed on housing 200 is also contemplated.

Rotation assembly 400 includes a rotation wheel 410 and a continuous rotation assembly 420. Rotation wheel 410 is engaged about a proximal end portion 102 of shaft 100 within housing 200 and extends outwardly through windows defined within either side of housing 200 to enable manual manipulation of rotation wheel 410. More specifically, rotation wheel 410 is continuously rotatable in either direction to thereby rotate shaft 100, and end effector 700 supported by shaft 100, relative to housing 200. Continuous rotation assembly 420 maintains electrical connection of jaw members 720, 760 of end effector 700 with the lead wires from cable 900 and/or activation assembly 800 regardless of the rotational orientation of rotation wheel 410 and shaft 100 relative to housing 200.

Trigger assembly 500 includes a trigger 510 and a plurality of linking structures 520 that operably connect trigger 510 to knife assembly 600 such that actuation of trigger 510 relative to housing 200 from an un-actuated position to an actuated position advances knife assembly 600 to thereby translate knife 610 of knife assembly 600 between jaw members 720, 760 to cut tissue grasped therebetween.

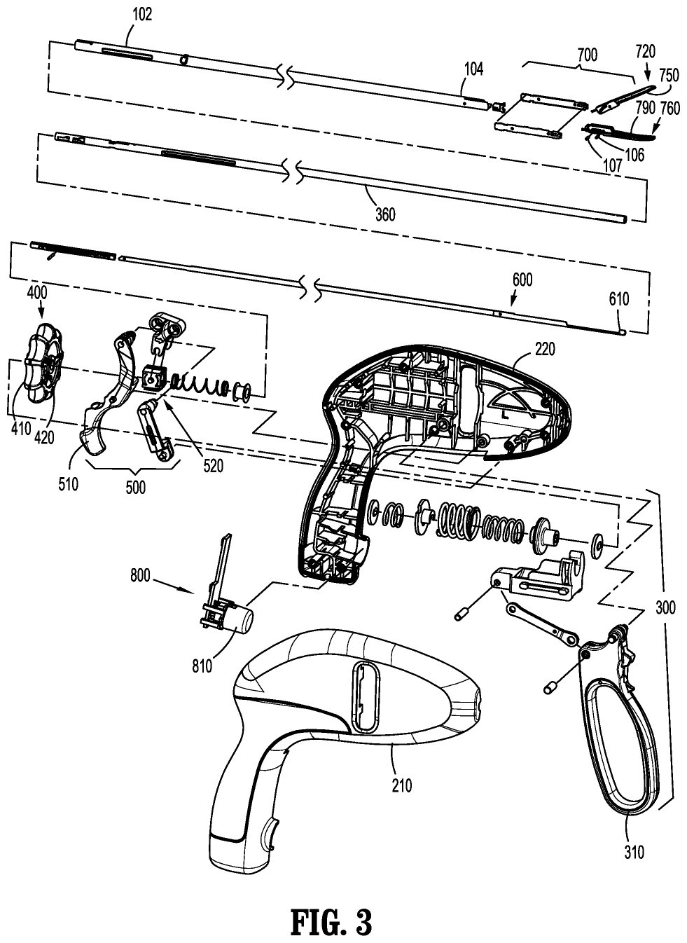

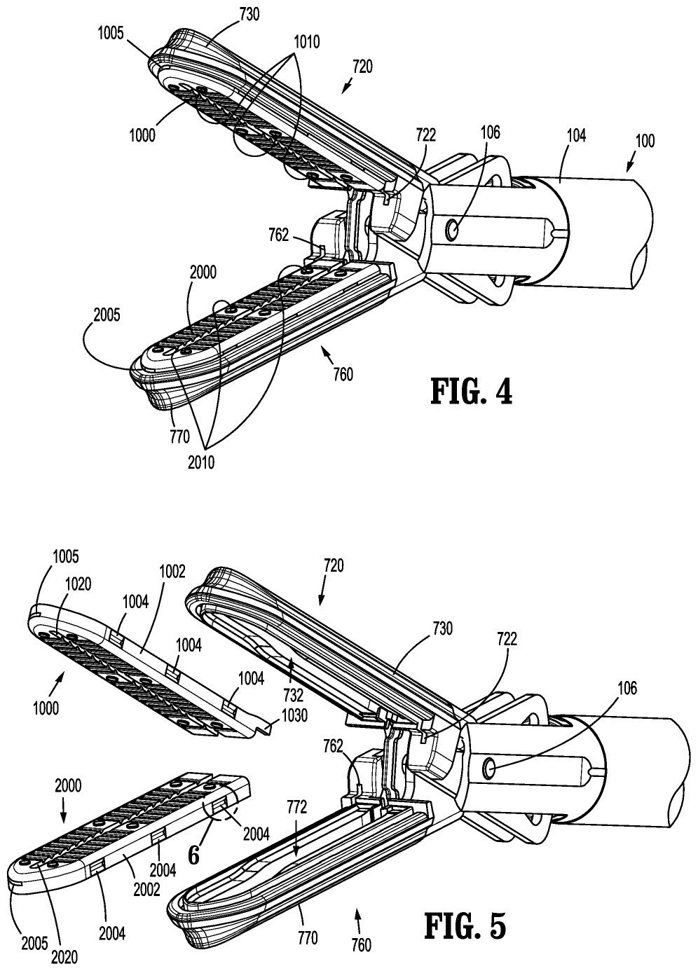

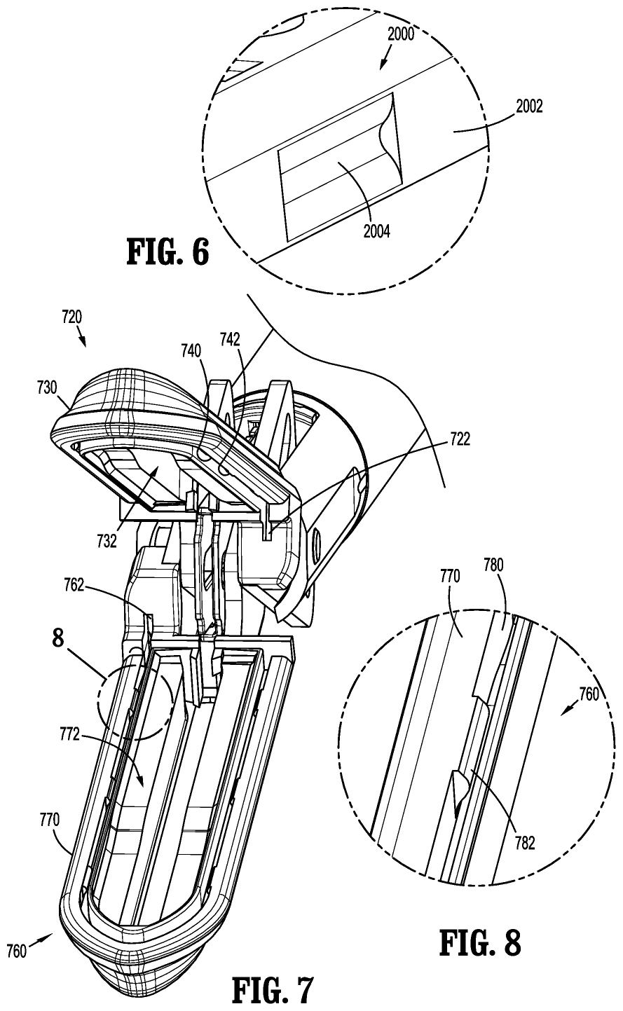

End effector 700 is supported at distal end portion 104 of shaft 100 and includes a first jaw member 720 and a second jaw member 760. A first replaceable seal plate 1000 is selectively engageable with the first jaw member 720, and a second replaceable seal plate 2000 is selectively engageable with the second jaw member 760 (FIGS. 4-16). Each replaceable seal plate 1000, 2000 includes an electrically conductive portion and is configured to contact tissue. A pivot pin 106 (e.g., FIGS. 3-5) pivotably couples jaw members 720, 760 with one another, while a cam pin 107 (FIG. 3) operably couples drive sleeve 360 to jaw members 720, 760. Thus, translation of drive sleeve 360 through shaft 100 in response to actuation of movable handle 310 relative to housing 200 translates cam pin 107 through cam slots of jaw members 720, 760 to thereby pivot jaw member 720 (or both jaw members 720, 760) relative to the other jaw member 760 between a spaced-apart position, where replaceable seal plates 1000, 2000 are farther apart from one another, and an approximated position, where replaceable seal plates 1000, 2000 are in closer approximation to one another to grasp tissue therebetween.

One or both replaceable seal plates 1000, 2000 may include a series of stop members 1010, 2010 (FIG. 4) configured to maintain a gap between replaceable seal plates 1000, 2000 and inhibit electrical shorting therebetween, e.g., by being formed from an insulative material or by being electrically isolated from one or both of the replaceable seal plates 1000, 2000. Replaceable seal plates 1000, 2000 include respective knife channel sections 1020, 2020 (FIG. 5) which cooperate to define a knife channel extending longitudinally therethrough when jaw members 720, 760 are in the approximated position to facilitate and guide reciprocation of knife 610 of knife assembly 600 (FIG. 3) through jaw members 720, 760 to cut tissue grasped therebetween.

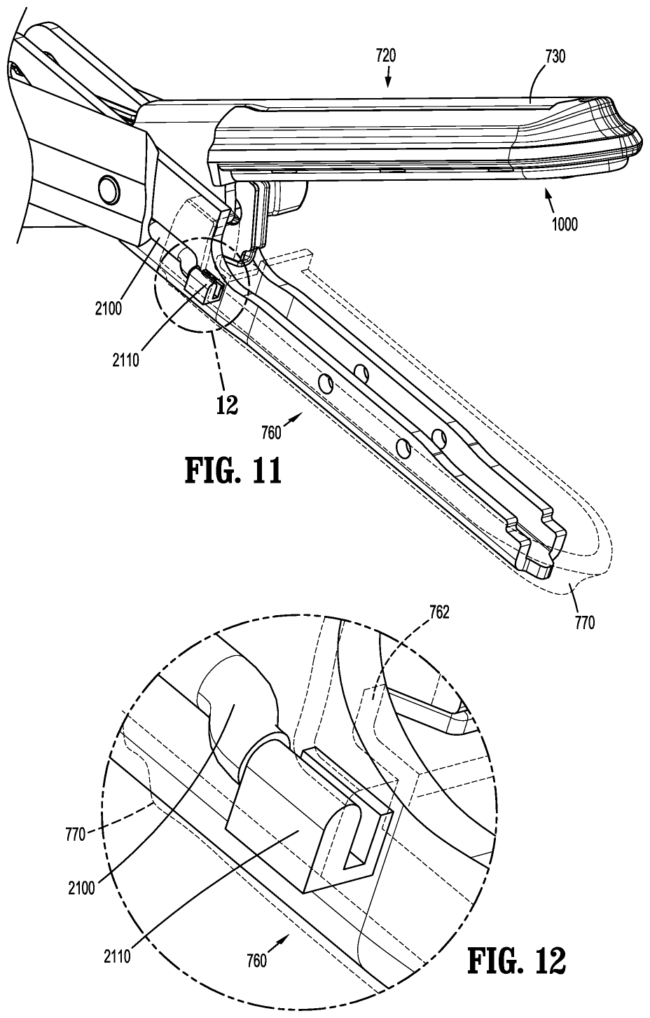

Additionally, jaw members 720, 760 include lead wires (collectively referred to by reference character 2100) that are adapted to connect to replaceable seal plates 1000, 2000 (for clarity, only lead wire 2100 associated with replaceable seal plate 2000 is shown; see FIGS. 11 and 12, for instance). It is further envisioned that surgical instrument 10 includes a single lead wire 2100 with bifurcated distal ends. Lead wires 2100 are adapted to connect replaceable seal plates 1000, 2000 to a source of electrosurgical energy, e.g., an electrosurgical generator (not shown), via cable 900, such that, upon activation, replaceable seal plates 1000, 2000 are energized to different potentials to enable the conduction of energy therebetween and through the grasped tissue to treat, e.g., seal, the grasped tissue.

Further aspects and features of surgical instrument 10, and more detail regarding the above-noted aspects and features of surgical instrument 10 can be found in U.S. patent application Ser. Nos. 17/122,062; 17/122,113; and Ser. No. 17/122,144, all filed on Dec. 15, 2020, the entire contents of each of which are hereby incorporated herein by reference. Additionally, while surgical instrument 10 is described herein as being energy-based, the surgical instrument 10 of the present disclosure may be other, non-energy-based instruments without departing from the scope of the present disclosure.

Referring to FIGS. 4-16, details of replaceable seal plates 1000, 2000 are shown. Generally, replaceable seal plates 1000, 2000 give a user the ability to remove a soiled or damaged seal plate from a jaw member, and replace the soiled seal plate with a new or cleaned seal plate during a surgical procedure.

As shown in FIG. 5, the first jaw member 720 includes an outer housing or shell 730, and the second jaw member 760 includes an outer housing or shell 770. Each shell 730, 770 includes a respective recess or cavity 732, 772 which accommodates respective replaceable seal plate 1000, 2000. More particularly, replaceable seal plate 1000 is configured to removably attach to shell 730 of first jaw member 720 such that a portion of replaceable seal plate 1000 is at least partially within cavity 732. Likewise, replaceable seal plate 2000 is configured to removably attach to shell 770 of second jaw member 760 such that a portion of replaceable seal plate 2000 is at least partially within cavity 772.

Jaw members 720, 760 and replaceable seal plates 1000, 2000 include various features that enable and/or facilitate the ability of replaceable seal plates 1000, 2000 to be removable from and attachable to respective jaw members 720, 760.

More particularly, and with reference to FIGS. 5-8, jaw members 720, 760 and replaceable seal plates 1000, 2000 include structure that enables a snap-fit connection therebetween, respectively. That is, each lateral side 1002 of first replaceable seal plate 1000, includes a plurality of grooves 1004 defined therein, and each lateral side 2002 of second replaceable seal plate 2000 includes a plurality of grooves 2004 defined therein. Additionally, inward-facing walls 740, 780 of shells 730, 770 of respective jaw members 720, 760 include a plurality of respective lips 742, 782 extending inwardly therefrom toward and/or into respective cavities 732, 772.

The grooves 1004 of the first replaceable seal plate 1000 are configured to mechanically engage the lips 742 of the first jaw member 720, and the grooves 2004 of the second replaceable seal plate 2000 are configured to mechanically engage the lips 782 of the second jaw member 760 (see FIG. 16).

Additionally, with particular reference to FIGS. 4 and 5, first replaceable seal plate 1000 includes a detent 1005 at its distal end, and second replaceable seal plate 2000 includes a detent 2005 at its distal end. Detents 1005, 2005 are configured to facilitate detachment of replaceable seal plates 1000, 2000 from respective shells 730, 770 of first jaw member 720 and second jaw member 760. That is, detents 1005, 2005 permit insertion of a tool (e.g., screwdriver, fingernail, etc.) at least partially therein to allow the tool to pry replaceable seal plate 1000, 2000 from respective shell 730, 770.

While the accompanying figures illustrate a particular number and size of the grooves 1004, 1005 and lips 742, 782, other amounts and sizes of the grooves 1004, 1005 and lips 742, 782 are contemplated by this disclosure.

With particular reference to FIGS. 4, 5, 7, 8, 11, 12, 14 and 15, each replaceable seal plate 1000, 2000 includes a respective finger 1030, 2030 extending proximally from a proximal end thereof (FIGS. 5, 8 and 15), and a distal end of lead wires 2100 includes a crimp contact 2110 engaged with each jaw member 720, 760 (FIGS. 11, 12 and 15). (The crimp contact associated with the first jaw member 720 is not clearly shown in the accompanying figures; both crimp contacts are collectively referred to herein by reference character 2110.) Additionally, each jaw member 720, 760 defines a respective slit 722, 762 adjacent crimp contact 2110 (FIGS. 4, 5, 7 and 14).

Each finger 1030, 2030 is configured to be inserted at least partially through respective slits 722, 762 of jaw member 720, 760, and into engagement with crimp contacts 2110. The engagement between the fingers 1030, 2030 and crimp contacts 2110 enables current to flow from lead wires 2100, through crimp contacts 2110, and to the replaceable seal plates 1000, 2000. Thus, when replaceable seal plates 1000, 2000 are engaged with crimp contacts 2110, replaceable seal plate 1000, 2000 are in electrical communication with the source of electrosurgical energy.

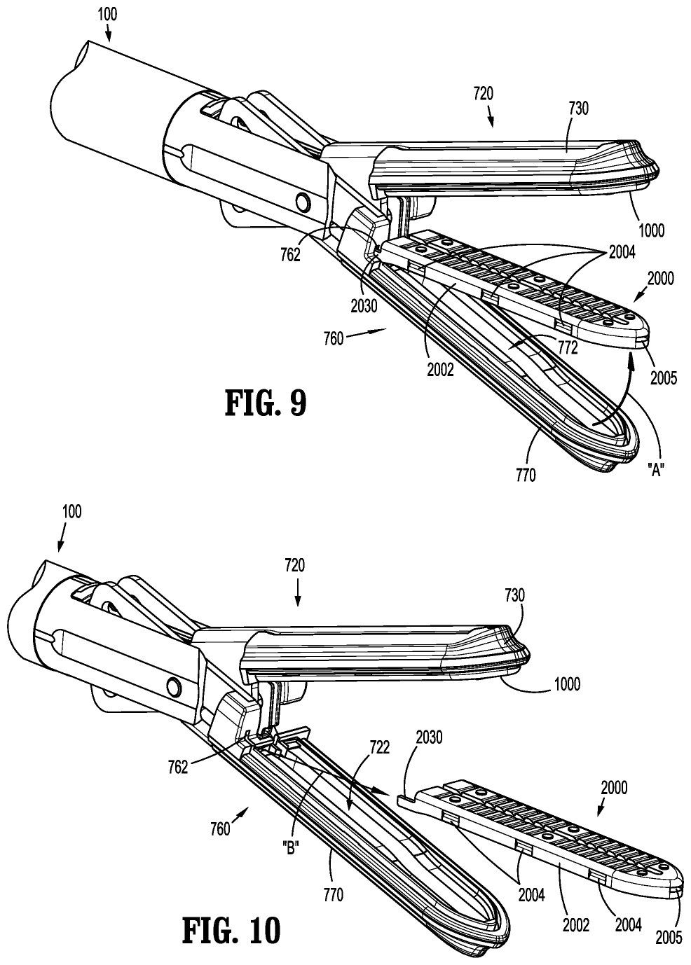

During use, a user is able to remove replaceable seal plates 1000, 2000 from mechanical and electrical engagement with respective jaw member 720, 760, and is able to position replaceable seal plates 1000, 2000 into mechanical and electrical engagement with respective jaw member 720, 760. More particularly, with reference to FIGS. 9 and 10, and focusing on a single replaceable seal plate for clarity and conciseness, to remove replaceable seal plate 2000 from engagement with jaw member 760, a user inserts a tool at least partially into detent 2005 and pivots replaceable seal plate 200 about finger 2030 relative to shell 770 in the general direction of arrow “A” in FIG. 9. As shown in FIG. 10, replaceable seal plate 2000 is then moved away from shell 770 in the general direction of arrow “B” which removes the finger 2030 of replaceable seal plate 2000 from crimp contact 2110.

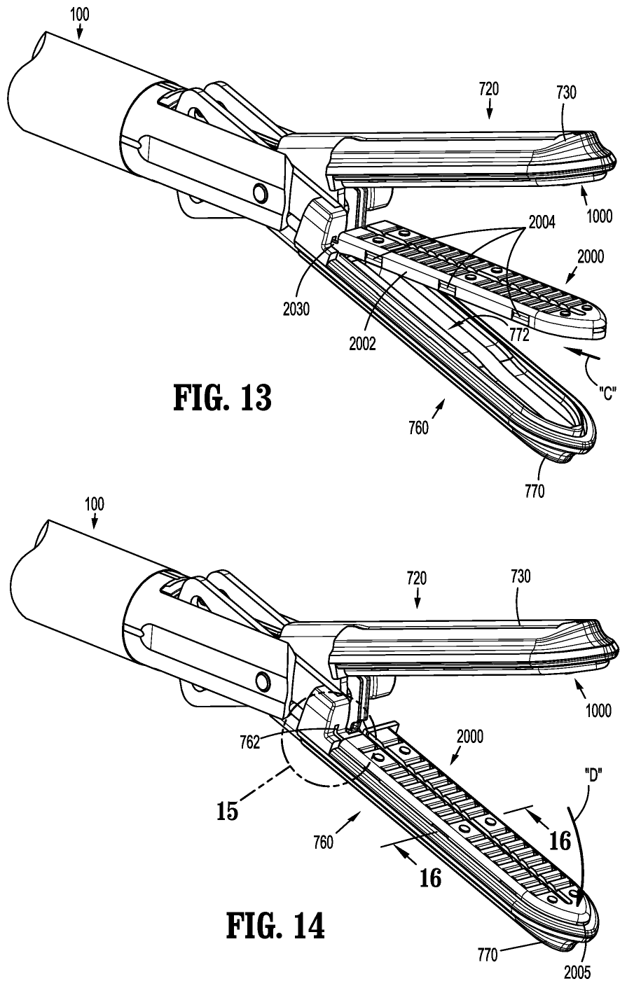

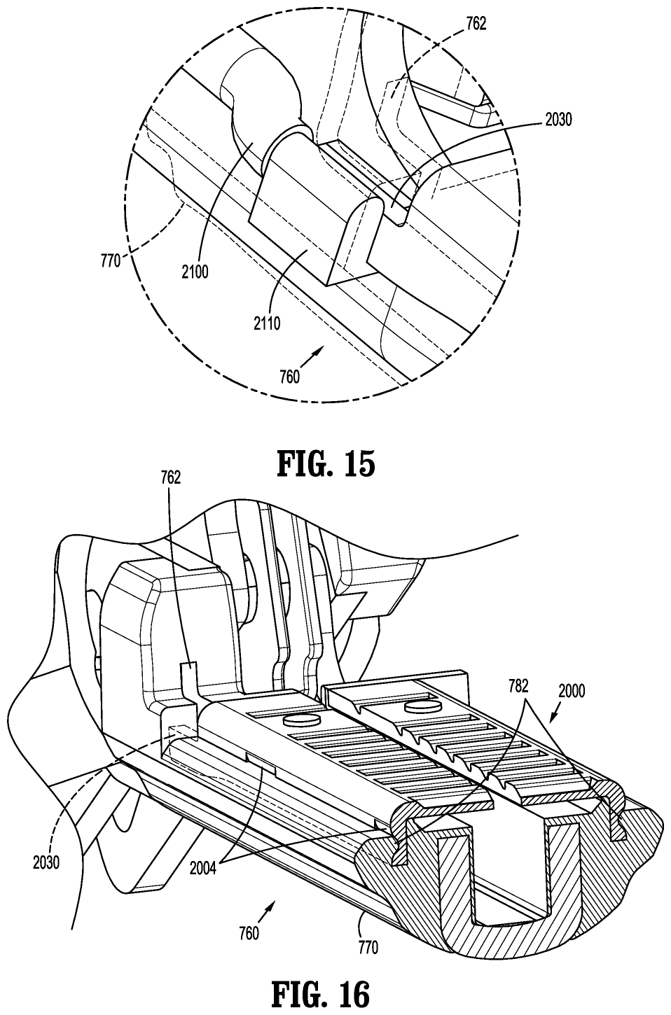

To insert replaceable seal plate 2000 into mechanical and electrical engagement with jaw member 760, a user moves replaceable seal plate 2000 in the general direction of arrow “C” in FIG. 13 relative to shell 770 such that finger 2030 is positioned at least partially within crimp contact 2110 (see also FIG. 15). Next, replaceable seal plate 2000 is pivoted about finger 2030 relative to shell 770 in the general direction of arrow “D” in FIG. 12. Here, the user is able to continue a surgical procedure or start a new surgical procedure.

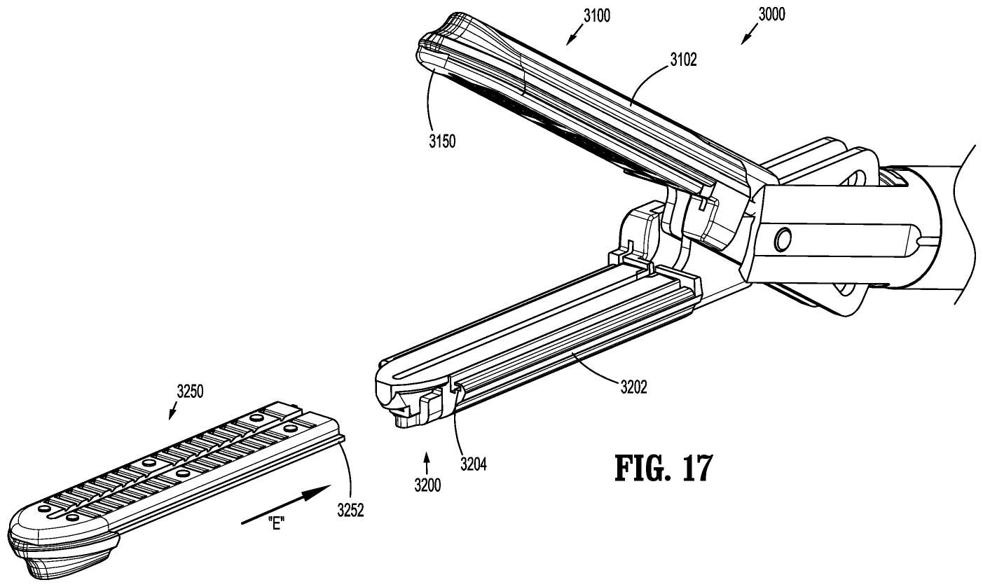

Referring now to FIGS. 17-21, a second embodiment of an end effector 3000 for use with surgical instrument 10 is shown. End effector 3000 includes a first jaw member 3100 having a first replaceable seal plate 3150, and a second jaw member 3200 having a second replaceable seal plate 3250. Many similarities exist between end effector 700 and end effector 3000; only differences between end effector 700 and end effector 3000 are discussed in detail below.

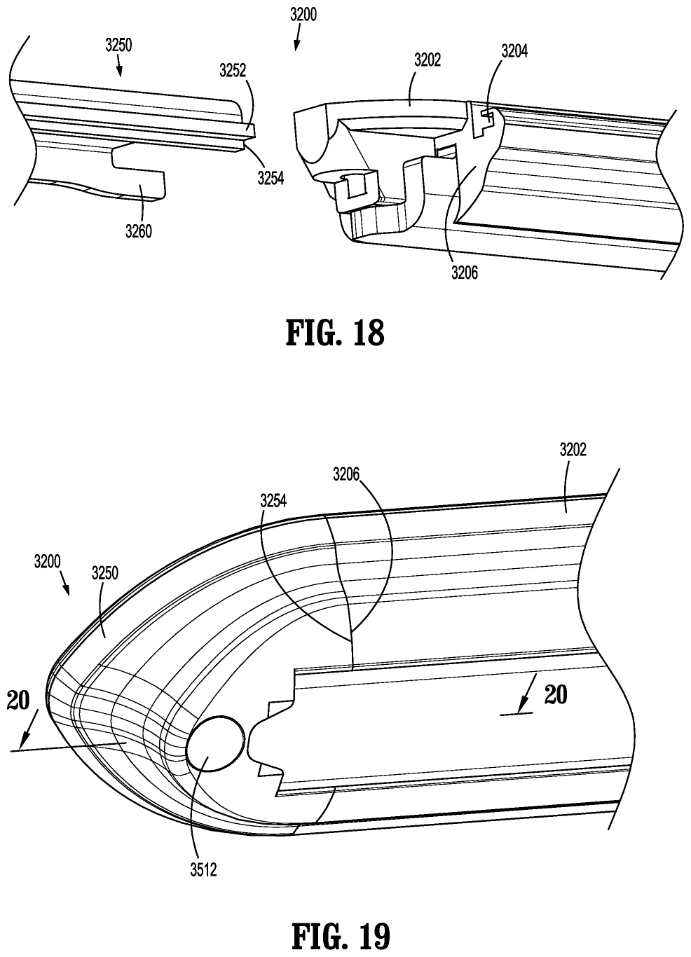

As shown in FIG. 17, the first jaw member 3100 includes an outer housing or shell 3102, and the second jaw member 3200 includes an outer housing or shell 3202. With reference to FIG. 18, shell 3202 includes a groove 3204 which is configured to accommodate a portion of second replaceable seal plate 3250. More particularly, second replaceable seal plate 3250 includes a tongue 3252 configured to slidingly engage groove 3204 of shell 3202 of second jaw member 3200.

While various features of second jaw member 3200 and second replaceable seal plate 3250 are described and illustrated herein, first jaw member 3100 and first replaceable seal plate 3150 also include these features, respectively, but such features omitted for clarity and brevity. Further, while only a first lateral side of shell 3202 and second replaceable seal plate 3250 is clearly visible in the accompanying figures, the second lateral side of shell 3202 and second replaceable seal plate 3250 includes the same features (e.g., a mirror-image) as the first lateral side thereof.

With continued reference to FIGS. 17 and 18, to insert second replaceable seal plate 3250 onto shell 3202 of second jaw member 3200, a user translates second replaceable seal plate 3250 in the general direction of arrow “E” in FIG. 17 relative to second jaw member 3200 a sufficient distance until tongue 3252 on each lateral side of second replaceable seal plate 3250 slidingly engages corresponding grooves 3204 of shell 3202 of second jaw member 3200. After engagement between tongues 3252 and grooves 3204, the user continues to move the second replaceable seal plate 3250 relative to second jaw member 3200 until a proximal-facing wall 3254 of second replaceable seal plate 3250 contacts a distal-facing wall 3206 of shell 3202 of second jaw member 3200, as shown in FIG. 19. Additionally, second replaceable seal plate 3250 includes a finger 3260 extending proximally from a proximal portion thereof and is configured to slidingly engage the first crimp contact 2110 (shown in FIG. 12, for instance) prior to second replaceable seal plate 3250 and shell 3202 reaching the fully engaged position (FIG. 19).

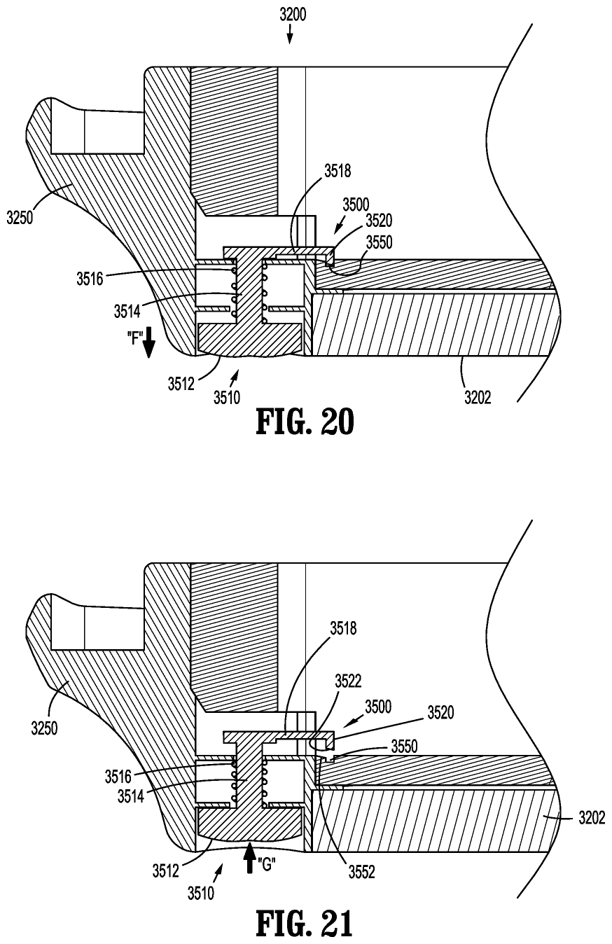

FIGS. 20 and 21 illustrate a locking mechanism 3500 configured to retain engagement between second replaceable seal plate 3250 and shell 3202 of second jaw member 3200. In FIG. 20, locking mechanism 3500 is engaged, thereby preventing relative movement between second replaceable seal plate 3250 and shell 3202. In FIG. 21, locking mechanism 3500 is disengaged, thereby enabling relative movement between second replaceable seal plate 3250 and shell 3202.

With continued reference to FIGS. 20 and 21, locking mechanism 3500 includes a button assembly 3510 disposed at least partially within second replaceable seal plate 3250, and a recess 3550 defined within shell 3202 of second jaw member 3200. Button assembly 3510 includes an outer surface 3512 configured to be engaged by a user, a stem 3514 extending from outer surface 3512, a biasing element 3516 surrounding at least a portion of stem 3514, a leg 3518 extending from stem 3514, and a toe 3520 extending from arm 3518. Toe 3520 of button assembly 3510 is configured to selectively engage recess 3550 of shell 3202 of second jaw member 3200.

Button assembly 3510 is movable between a first position (FIG. 20), where toe 3520 is engaged with recess 3550, and a second position (FIG. 21), where toe 3520 is spaced from recess 3550. In the first position, second replaceable seal plate 3250 is engaged with, and therefor immovable with respect to shell 3202 of second jaw member 3200. In the second position, second replaceable seal plate 3250 is movable (i.e., distally) relative to shell 3202 of second jaw member 3200. Biasing element 3516 biases outer surface 3512 of button assembly 3510 in the general direction of arrow “F” in FIG. 20 such that toe 3520 is engaged with recess 3550. Depressing outer surface 3512 of button assembly 3510 in the general direction of arrow “G” in FIG. 21 causes finger 3520 to disengage from recess 3550.

An angled surface 3522 on toe 3520 and/or an angled surface 3552 adjacent recess 3550 facilitate engagement and/or disengagement between of second replaceable seal plate 3250 relative to shell 3202 of second jaw member 3200.

While illustrated as being used in one type of hand-held surgical instrument, it is contemplated, and within the scope of the present disclosure for the replaceable seal plates 1000, 2000, 3150, 3250 to be configured for use with various electromechanical and/or electrosurgical instruments and systems. For example, the replaceable seal plates 1000, 2000, 3150, 3250 may be utilized in robotic surgical systems, such as the robotic surgical system shown and described in U.S. Pat. No. 8,828,023, the entire content of which is incorporated herein by reference.

While several configurations of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical instrument, comprising:
a housing;
a shaft extending distally from the housing;
an end effector supported adjacent a distal end of the shaft and including a first jaw member and a second jaw member, at least one of the first jaw member or the second jaw member movable relative to the other between an open position and an approximated position;
a lead wire extending at least partially through the shaft and adapted to connect to a source of electrosurgical energy; and
a first replaceable seal plate configured for selective engagement with the first jaw member and configured for selective engagement with the lead wire, the first replaceable seal plate including an electrically conductive portion and configured for contacting tissue when engaged with the first jaw member.

2. The surgical instrument according to claim 1, wherein a first distal end of the lead wire includes a first crimp contact.

3. The surgical instrument according to claim 2, wherein a finger extending proximally from a proximal portion of the first replaceable seal plate is configured for pivotal engagement with the first crimp contact.

4. The surgical instrument according to claim 1, wherein the first jaw member includes a shell defining a cavity, the first replaceable seal plate selectively positionable at least partially within the cavity.

5. The surgical instrument according to claim 4, wherein the shell of the first jaw member includes a plurality of lips configured to engage the first replaceable seal plate.

6. The surgical instrument according to claim 5, wherein the first replaceable seal plate includes a plurality of grooves, each lip of the plurality of lips of the shell of the first jaw member configured to engage one groove of the plurality of grooves of the first replaceable seal plate.

7. The surgical instrument according to claim 6, wherein at least one groove of the plurality of grooves of the first replaceable seal plate is defined within a first lateral side of the first replaceable seal plate.

8. The surgical instrument according to claim 7, wherein at least one groove of the plurality of grooves of the first replaceable seal plate is defined within a second lateral side of the first replaceable seal plate.

9. The surgical instrument according to claim 8, wherein a distal end of the first replaceable seal plate defines a detent configured to facilitate disengaging the first replaceable seal plate from the first jaw member.

10. The surgical instrument according to claim 1, further comprising a second replaceable seal plate configured for selective engagement with the second jaw member and configured for selective engagement with the lead wire.

11. The surgical instrument according to claim 10, wherein a second distal end of the lead wire includes a second crimp contact.

12. The surgical instrument according to claim 11, wherein a proximal portion of the second replaceable seal plate is configured for pivotal engagement with the second crimp contact.

13. The surgical instrument according to claim 2, wherein the first jaw member includes a shell, the shell defining a slit adjacent the first crimp contact, a finger extending proximally from a proximal portion of the first replaceable seal plate is configured to selectively extend at least partially through the slit.

14. An end effector for use with a surgical instrument, the end effector comprising:
a first jaw member;
a second jaw member, at least one of the first jaw member or the second jaw member movable relative to the other between an open position and an approximated position;
a lead wire disposed in mechanical cooperation with the first jaw member and the second jaw member, and adapted to connect to a source of electrosurgical energy;
a first replaceable seal plate configured for selective engagement with the first jaw member and configured for selective engagement with the lead wire; and
a second replaceable seal plate configured for selective engagement with the second jaw member and configured for selective engagement with the lead wire.

15. The end effector according to claim 14, wherein a first distal end of the lead wire includes a first crimp contact associated with the first jaw member, and a second distal end of the lead wire includes a second crimp contact associated with the second jaw member.

16. The end effector according to claim 15, wherein a finger extending proximally from a proximal portion of the first replaceable seal plate is configured for pivotal engagement with the first crimp contact.

17. The end effector according to claim 16, wherein a finger extending proximally from a proximal portion of the second replaceable seal plate is configured for pivotal engagement with the second crimp contact.

18. The end effector according to claim 14, wherein the first replaceable seal plate is configured to selectively engage the first jaw member with a snap-fit connection, and the second replaceable seal plate is configured to selectively engage the second jaw member with a snap-fit connection.

19. The end effector according to claim 18, wherein a first lateral wall of the first replaceable seal plate and a first lateral wall of the second replaceable seal plate each include a groove, the first jaw member and the second jaw member each include at least one lip, the lip of the first jaw member configured to selectively engage the groove of the first replaceable seal plate, the lip of the second jaw member configured to selectively engage the groove of the second replaceable seal plate.

20. The end effector according to claim 19, wherein a distal end of the first replaceable seal plate defines a first detent configured to facilitate disengaging the first replaceable seal plate from the first jaw member, a distal end of the second replaceable seal plate defines a second detent configured to facilitate disengaging the second replaceable seal plate from the second jaw member.
